(19) United States
(12) Patent Application Publication
(10) Pub. No.: US 2011/0040316 A1
(43) Pub. Date: Feb. 17, 2011

(54) Method and Apparatus for a Single Handed Squeeze Lancet

(75) Inventors: James V. Pottala, Sioux Falls, SD (US); William S. Harris, Sioux Falls, SD (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: SANFORD RESEARCH/USD, Sioux Falls, SD (US)

(21) Appl. No.: 12/857,694

(22) Filed: Aug. 17, 2010

Related U.S. Application Data
(60) Provisional application No. 61/234,382, filed on Aug. 17, 2009.

(51) Int. Cl. A61B 17/34

(52) U.S. Cl. 606/182

(57) ABSTRACT

A single handed squeeze lancet and methods for its use, where the single handed squeeze lancet comprises (a) a lancet, (b) a lancet-holder, (c) an internal housing for receiving and guiding the lancet-holder, (d) an external housing for receiving and guiding the internal housing, (e) a thumb-frame coupled to the external housing, and (f) a thumb-spring coupled to the thumb-frame and coupled to the lancet-holder. The device may further comprise a base-stand coupled to the thumb-frame.

This application is a non-provisional of U.S. Provisional Application No. 61234382 for Method and Apparatus for a Single Handed Squeeze Lancet, filed Aug. 17, 2009.

### BACKGROUND OF THE INVENTION

Some diseases, such as diabetes, require that an individual test their blood on a daily basis. As a result, lancet devices are needed that pierce a patient's skin to obtain an amount of blood sufficient for testing. Typical blood collection lancets require the patient to lance a finger by holding a lancet in one hand and pressing it onto the fingertip on the other hand. Oftentimes the anticipation of the impending pain causes the patient to involuntarily pull the fingertip back at the last second. This often results in a poor stick, insufficient blood collection, and a failed test; if only one lancet is included in a mailed blood collection kit, the kit thus becomes useless.

### SUMMARY OF THE INVENTION

The present invention provides a single handed squeeze lancet apparatus that will not discharge unless the pressure from a patient's digit is sufficient to properly lance the digit. The single handed squeeze lancet has the additional benefit of guarding against accidental injuries after use by retaining the used lancet within the housing. The present invention further provides methods for use of the single handed squeeze lancet.

Thus, in a first aspect, the present invention provides a single handed squeeze lancet apparatus comprising: (a) a lancet, (b) a lancet-holder, (c) an internal housing for receiving and guiding the lancet-holder, (d) an external housing for receiving and guiding the internal housing, (e) a thumb-frame coupled to the external housing, and (f) a thumb-spring coupled to the thumb-frame and coupled to the lancet-holder.

In one embodiment, the invention provides a thumb-frame in the form of a base, such that the single handed squeeze lancet is employed between a digit and a surface.

In another embodiment, the invention further comprises a base-stand.

In a second aspect, the present invention also provides a method for lancing a digit using the single handed squeeze lancet apparatus, where the method comprises: (a) placing a digit adjacent to a proximal end of the internal housing and (b) applying force to the proximal end of the internal housing.

In one embodiment the method further comprises the steps of placing a digit from one of the same hand or the other hand adjacent to the thumb-frame and applying force to the thumb-frame.

In another embodiment, the method further comprises the steps of providing the thumb-frame in the form of a base and placing the thumb-frame against a surface.

In still another embodiment, the method further comprises the steps of providing a base-stand, removing the base-stand from a coupling to one of the thumb-frame or the external housing, coupling the base stand to one of the thumb-frame or the external housing to form a base, and placing the base against a surface.

### BRIEF DESCRIPTION OF THE DRAWINGS

FIG. 1 is an isometric exploded view of the single handed squeeze lancet apparatus.

FIG. 2 is a cross-sectional view of the fully assembled single handed squeeze lancet apparatus.

FIG. 3 is a front view of the fully assembled single handed squeeze lancet apparatus, including the base-stand.

FIG. 4 is an isometric view of the fully assembled single handed squeeze lancet apparatus, including the base-stand.

FIG. 5A is a front view of the external housing.

FIG. 5B is a side view of the external housing.

FIG. 5C is a cross-sectional view of the external housing.

FIG. 5D is an isometric view of the external housing.

FIG. 5E is a top view of the external housing.

FIG. 6A a front view of the internal housing.

FIG. 6B is a side view of the internal housing.

FIG. 6C is a cross-sectional view of the internal housing.

FIG. 6D is an isometric view of the internal housing.

FIG. 6E is a cross-sectional view of the internal housing.

FIG. 6F is a top view of the internal housing.

FIG. 7A is a front view of the lancet-holder and pull-tab.

FIG. 7B is a side view of the lancet-holder.

FIG. 7C is a front view of the lancet-holder and uncovered lancet.

FIG. 7D is an isometric view of the lancet-holder and pull-tab.

FIG. 7E is a top view of the lancet-holder.

FIG. 8A is a top view of the thumb-frame.

FIG. 8B is a front view of the thumb-frame and thumb-spring.

FIG. 8C is a side view of the thumb-frame and thumb-spring.

FIG. 8D is an isometric view of the thumb-frame and thumb-spring.

FIG. 9A is front view of the base-stand.

FIG. 9B is an isometric view of the base-stand.

FIG. 10 is a cross-sectional front view of the fully assembled single handed squeeze lancet apparatus in a storage or shipping position.

FIG. 11 is a cross-sectional front view of the fully assembled single handed squeeze lancet apparatus after activation.

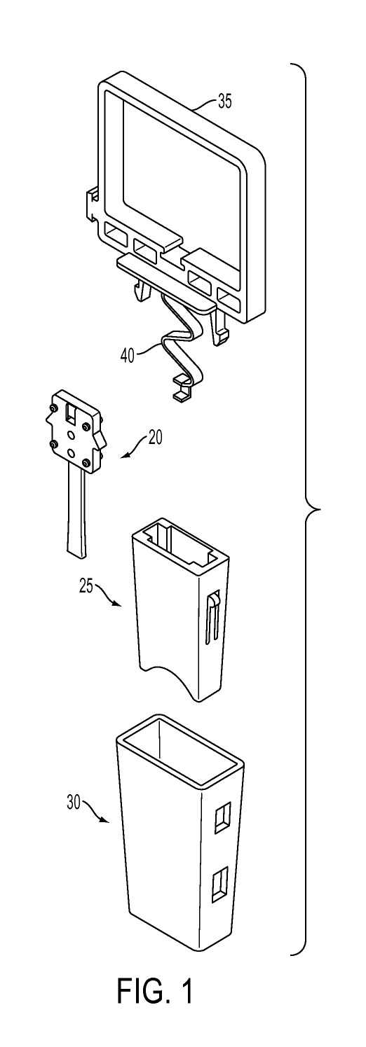

### DETAILED DESCRIPTION OF THE INVENTION

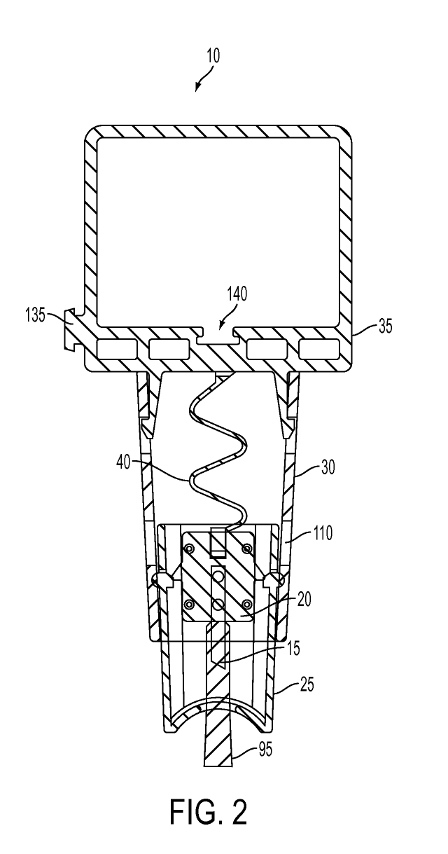

In a first aspect, as shown in FIG. 2, the present invention may take the form of a single handed squeeze lancet apparatus 10 comprising: (a) a lancet 15, (b) a lancet-holder 20, (c) an internal housing 25 for receiving and guiding the lancet-holder 20, (d) an external housing 30 for receiving and guiding the internal housing 25, (e) a thumb-frame 35 coupled to the external housing 30, and (f) a thumb-spring 40 coupled to the thumb-frame 35 and coupled to the lancet-holder 20.

As used herein, a “single handed squeeze lancet apparatus” can range in length from 0.5 to 7 inches, and is preferably in the range of 1.5 to 3 inches in length, which accommodates a typical span between digits on a single hand, as well as variances in the size of hands among men, women, and children. A preferred length of 2 also allows for peak grip force for both men and women.

As used herein, a “lancet” 15 is a sharp metal blade or small surgical instrument, usually a short, wide, sharp-pointed, double-edged blade, used especially for making punctures and small incisions in a person's skin. The proximal end 15a of the lancet 15 is the piercing end, while the distal end of the lancet 15 may define one or more holes that aid in coupling the lancet 15 to the lancet-holder 20.

As used herein, a “lancet-holder” 20 receives and secures the distal end of the lancet 15 to maintain the lancet's proper orientation within the single handed squeeze lancet apparatus 10. The lancet-holder 20 also acts as an interface with the internal housing 25 and thumb-spring 40 as described in more detail below. The lancet-holder 20 comprises a housing 45 that is preferably overmolded about the distal end of a medically clean lancet 15. Alternatively, the lancet-holder's housing could be pre-molded in two halves that contain male and female components that allow the two halves to be press-fit together. In this pre-molded embodiment, the one or more holes in the distal end of the lancet 15 receive corresponding male components molded on the lancet-holder housing 45 to secure the distal end of the lancet 15 within the lancet-holder 20. The lancet's distal end could also be affixed in a recess in the lancet-holder 20 via a common fastener such as rivets or small screws, for example, or via an adhesive. Alternatively, the lancet 15 may be press-fit into a slot in a one-piece lancet-holder 20. The lancet-holder 20 is preferably made of an injection molded thermoplastic, for example Polyamide (Nylon), ABS, Acetal, Polycarbonate (PC), Polyethylene (PE), Polypropylene (PP), or Polyvinyl chloride (PVC), which may further be blended or glass reinforced, though any other suitable material, such as a lightweight plastic or metal that is capable of being machined, is contemplated.

As used herein, an “internal housing” 25 preferably defines two opposing sidewalls 50a,b of substantially equal dimension in length, width and wall thickness. The length is in the range of 0.5 up to 7 inches and is preferably in the range of 0.5 to 1.5 inches. The width is in the range of 0.08 to 1 inch and is preferably in the range of 0.15 up to 0.5 inches. The wall thickness is in the range of 0.01 up to 0.3 inches and is preferably in the range of 0.02 to 0.12 inches. The internal housing 25 also defines a front wall 55 and a back wall 60 of substantially equal dimension in length, width and thickness. The length is in the range of 0.5 up to 7 inches and is preferably in the range of 0.5 to 1.5 inches. The width is in the range of 0.25 up to 3 inches and is preferably in the range of 0.4 to 2 inches. The wall thickness is in the range of 0.01 up to 0.3 inches and is preferably in the range of 0.02 to 0.12 inches. The opposing sidewalls 50a,b can be of different dimensions than the front and back walls 55, 60. These four walls further define an opening at both the distal end 25b and proximal end 25a of the internal housing 25. In assembly, the lancet-holder 20 is received in the internal housing 25 via its distal opening and in use the lancet 15 will discharge through the internal housing's proximal opening. The internal housing 25 interfaces with both the external housing 30 and the lancet-holder 20, as described in more detail below. The internal housing 25 is preferably made of ABS, though any other suitable material, such as another lightweight plastic or metal, is contemplated.

As used herein, an “external housing” 30 preferably defines two opposing sidewalls 65a,b of substantially equal dimension in height, length, width and thickness. The length is in the range of 0.5 to 7 inches and is preferably in the range of 0.75 to 2 inches. The width is in the range of 0.08 to 1 inch and is preferably in the range of 0.15 to 0.5 inches. The wall thickness is in the range of 0.01 to 0.3 inches and is preferably in the range of 0.02 to 0.12 inches. The external housing 30 also defines a front wall 70 and a back wall 75 of substantially equal dimension in height, length, width and thickness. The length is in the range of 0.5 to 7 inches and is preferably in the range of 0.75 to 2 inches. The width is in the range of 0.25 to 3 inches and is preferably in the range of 0.5 to 2 inches. The wall thickness is in the range of 0.01 to 0.3 inches and is preferably in the range of 0.02 to 0.12 inches. The opposing sidewalls 65a,b can be of different dimensions than the front and back walls 70, 75. These four walls further define an opening at both the distal end 30b and proximal end 30a of the external housing 30. The external housing 30 is dimensioned to circumscribe the internal housing 25 to substantially prevent the internal housing 25 from rotating and to guide the internal housing 25 during assembly and operation of the single handed squeeze lancet apparatus 10. In assembly, the internal housing 25, the lancet-holder 20, and the thumb-spring 40 are received in the external housing 30 via its distal opening, as described in more detail below. Post-assembly, a predetermined length of the internal housing 25 protrudes from the proximal opening of the external housing 30. The external housing 30 is preferably made of ABS, though any other suitable material, such as another lightweight plastic or metal, is contemplated.

As used herein, a “thumb-frame” 35 is coupled to the external housing 30 at or near the housing's distal end. The thumb-frame 35 preferably defines a ledge 80 that either covers or nests within the distal end 30b of the external housing 30, sealing the internal housing 25, lancet-holder 20, lancet 15, and thumb-spring 40 within the external housing 30. For reasons discussed in detail below, the thumb-frame 35 preferably defines a square or rectangular outer profile for receiving a digit on a hand. In addition, regardless of the shape of the thumb-frame's outer profile, the portion of the thumb-frame's inner profile that receives a digit may take the form of a flat surface or an arcuate notch. Alternatively, the thumb-frame 35 may only partially border the digit (not shown), preventing the digit from slipping off the distal end of the lancet apparatus 10. This partial thumb-frame (not shown) could take the form of an Omega, a U-shape, or an arcuate notch, for example. The thumb-frame's inner profile accommodates a diameter ranging from 0.5 to 2 inches, preferably the diameter is 1 inch. Diameters larger than 1 inch, however, allow more than one digit to be employed in the thumb-frame 35. The thumb-frame 35 is preferably made of an injection molded thermoplastic, for example Polyamide (Nylon), ABS, Acetal, Polycarbonate (PC), Polyethylene (PE), Polypropylene (PP), or Polyvinyl chloride (PVC), which may further be blended or glass reinforced, though any other suitable material, such as a lightweight plastic or metal that is capable of being machined, is contemplated.

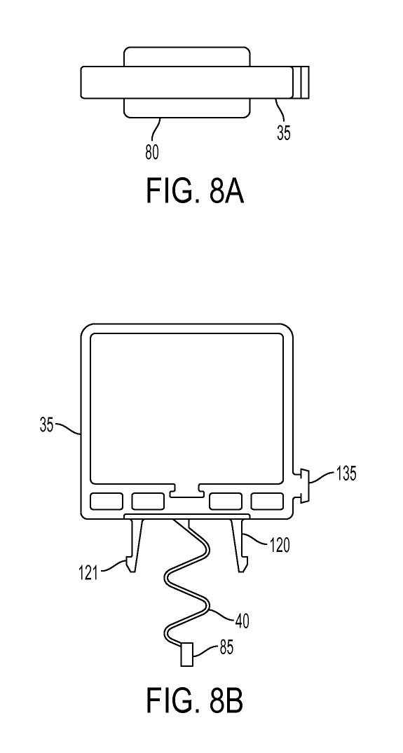

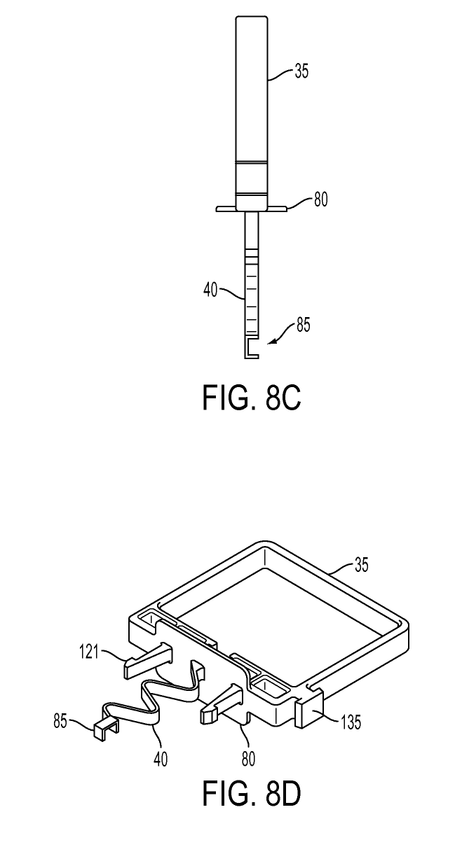

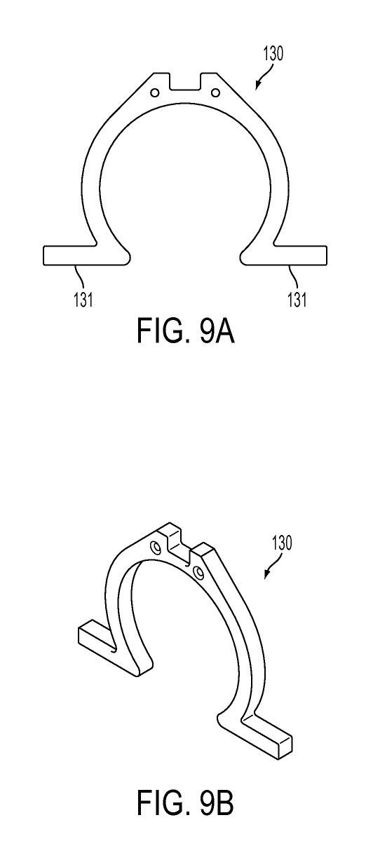

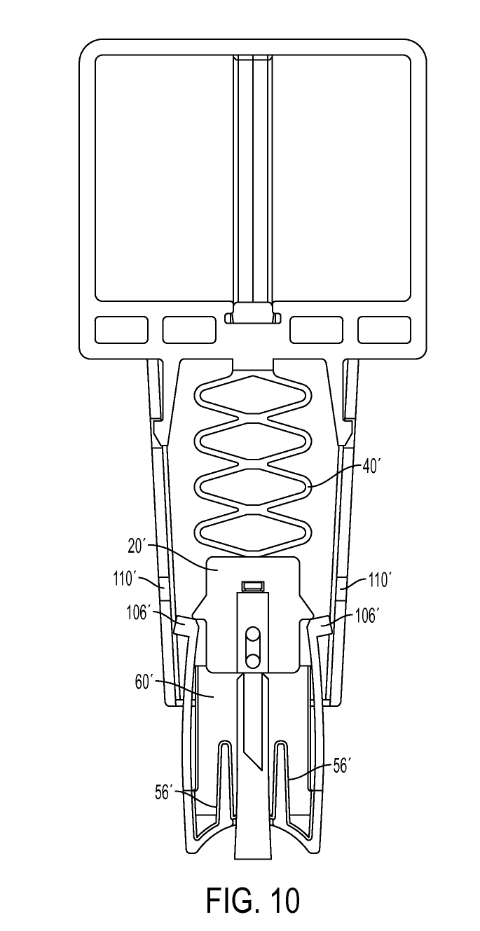

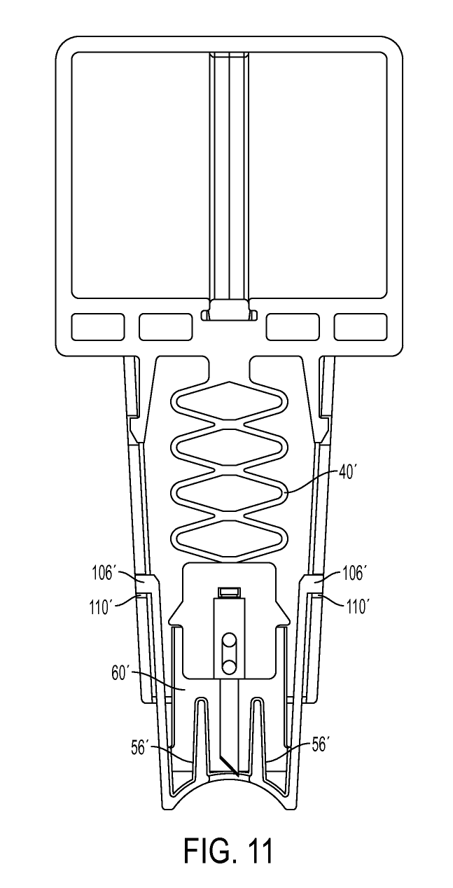

As used herein, a “thumb-spring” 40 has a distal end coupled to the thumb-frame 35 and a proximal end coupled to the lancet-holder 20. The thumb-spring 40 is preferably integrally formed with the thumb-frame 35 using conventional molding techniques, and the thumb-spring's proximal end is integrally formed into a substantially U-shaped coupling member 85. This coupling member is received in a corresponding recess or channel 90 in the lancet-holder's housing. Alternatively, the thumb-spring's proximal end may comprise a post that is received in a corresponding hole in the lancet holder's housing 45. Further, the thumb-spring 40 may be snap-fit to the lancet holder's housing 45 or the thumb-spring 40 and lancet holder housing 45 could be molded as one piece. Still alternatively, the thumb frame 35, thumb spring 40 and the entire lancet holder assembly 20 may be molded as one piece. The thumb-spring 40 is preferably made of an injection molded thermoplastic, for example Polyamide (Nylon), ABS, Acetal, Polycarbonate (PC), Polyethylene (PE), Polypropylene (PP), or Polyvinyl chloride (PVC), which may further be blended or glass reinforced, though any other suitable material, such as a lightweight plastic or metal that is capable of being machined, is contemplated. In this form, the thumb-spring may comprise a sinusoidal spring 40, as shown in FIGS. 2 and 8A-D, or may comprise 2 to 4 symmetric, stacked loops 40′, as shown in FIGS. 10-11. Alternatively, the thumb-spring 40 may take the form of a helical or conical compression spring, for example.

In one embodiment, the lancet-holder 20 comprises a housing 45, at least two guide pins 46, and at least two detents 47. The lancet-holder 20 is dimensioned to be received within the internal housing 25. Similar to the internal housing 25, the lancet-holder housing 45 defines opposing sidewalls of substantially equal dimension in length, width, height, and thickness, as well as a front wall and a back wall of substantially equal dimension in length, width, height, and thickness. Also note, the lancet-holder housing's sidewalls can be of different dimension than the front and back walls of the lancet-holder housing 45. The front and back walls of the lancet-holder housing 45 preferably define a rectangular profile, though different profiles are contemplated, for example, an oval, a triangle, or a hexagon.

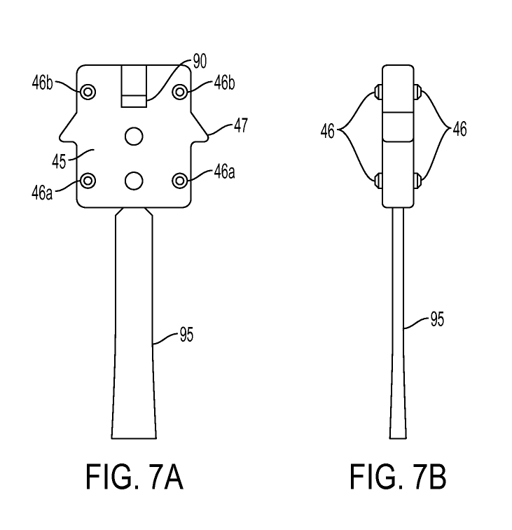

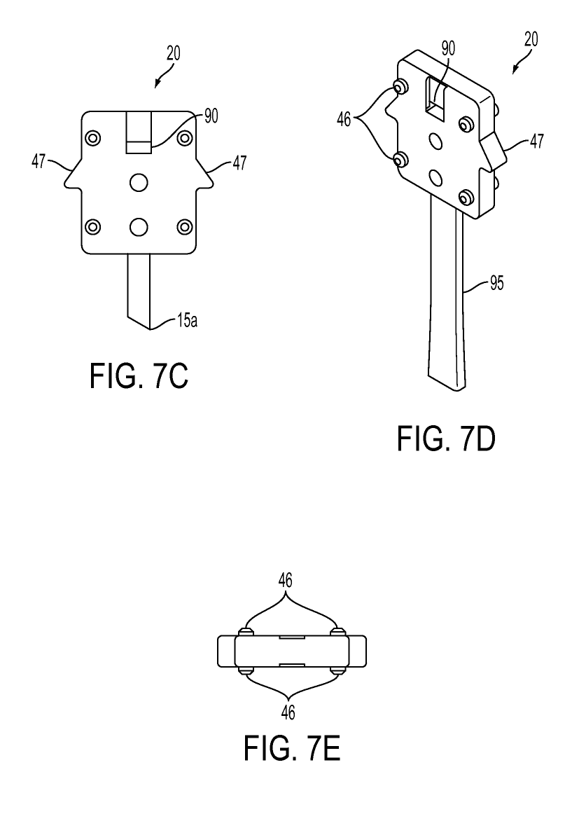

The guide pins 46 are preferably located on both the front and back walls of the lancet-holder housing 45. There are preferably four guide pins on the front wall arranged symmetrically as if each guide pin were located at the corner of a square or rectangle. The preferred arrangement results in a proximal pair of guide pins 46a and a distal pair of guide pins 46b. As shown in FIG. 7B, the guide pins on the back wall are preferably arranged such that each guide pin substantially shares the same axis as the corresponding guide pin on the front wall. Alternatively, any number or arrangement of guide pins could be employed on both the front and back walls, such as a single pair of guide pins located somewhere between the proximal and distal ends of the lancet-holder 20. Another example is a single pair of ribs orthogonal to the front and back walls that are substantially parallel to the longitudinal axis of the lancet 15. Additionally, a single guide pin may be located on each of the front and back walls 55, 60. Further, there are preferably two detents 47 that are located opposite one another on the opposing sidewalls of the lancet-holder housing 45. The guide pins 46 and detents 47 are preferably integrally formed with the lancet-holder housing 45 using conventional molding techniques.

In another preferred embodiment, the lancet-holder 20 may further comprise a pull-tab 95, as shown in FIG. 7A. As used herein, the pull-tab 95 is used to protect the lancet 15 against contamination, oxidation, and inadvertent lancing. Prior to use, the pull-tab 95 is simply pulled by the user to expose the lancet 15 within the apparatus 10. The pull-tab 95 may be integrally molded as part of the lancet-holder housing 45 or may be a separate covering. A pull-tab 95 is unnecessary if the apparatus 10 is hermetically-sealed at the time of assembly, for example, in heat-sealed packaging.

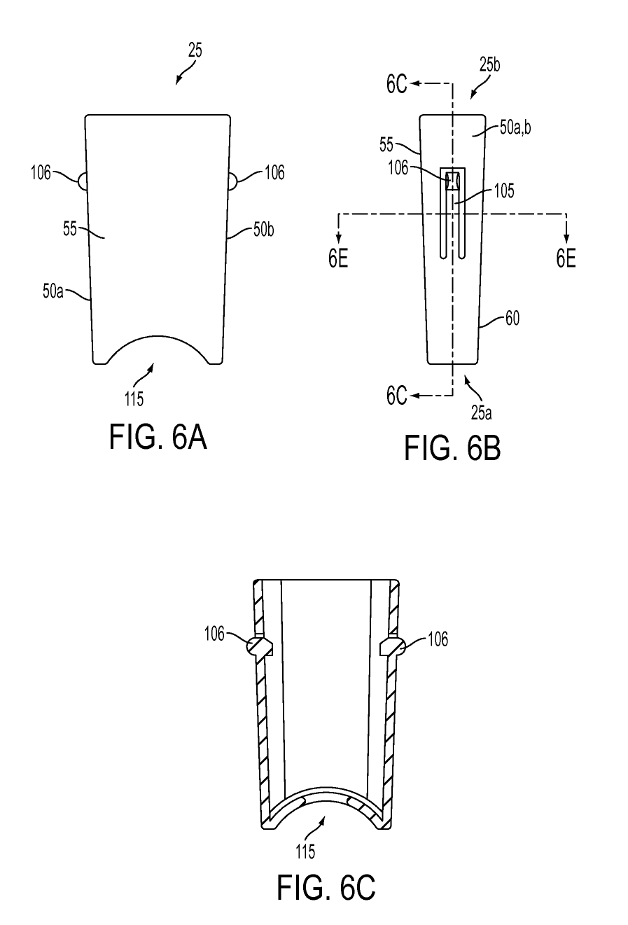

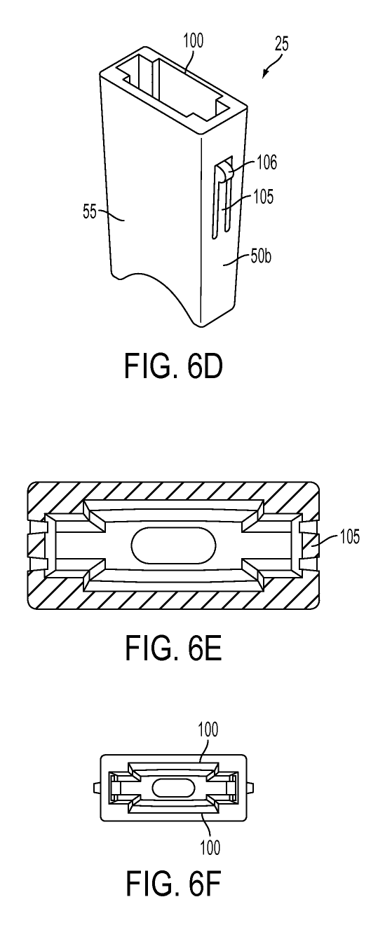

In one preferred embodiment, shown in FIGS. 6C-F, the internal housing 25 defines channels 100 for receiving the lancet-holder guide pins 46. The front and back walls 55, 60 of the internal housing 25 each preferably define a single channel 100 beginning at the distal end of the internal housing 25 and terminating near the proximal end of the internal housing 25 above the proximal opening. The channel 100 is wide enough to accommodate a pair of guide pins 46. Alternatively, the front and back walls may define multiple channels, such that the width of each channel will accommodate a single guide pin 46. Thus, during assembly and operation of the lancet apparatus 10, the channels of the internal housing 25 help guide the lancet-holder 20 and help maintain the lancet's proper alignment within the apparatus 10.

In another preferred embodiment, shown in FIGS. 10-11, in the shipping and storage position at least a portion of the proximal end of the lancet holder 20 extends between the front and back walls 55, 60 of the internal housing and/or between guiding ribs (not shown) that extend distally from the front and back walls 55, 60. In this embodiment, the lancet holder 20 is approximately 0.005 to 0.25 inches thinner than the cavity defined between the front and back walls 55, 60 of the internal housing 25, which allows the lancet to advance within the internal housing 25 in the absence of guide pins 46 and/or channels 100.

In one preferred embodiment, the internal housing 25 includes tongues or cantilever arms 105 on opposing sidewalls, wherein each internal-housing tongue 105 includes an interference nub 106 on a free end. As used herein, an interference nub 106 is a knob or protuberance, preferably integrally molded with the internal-housing tongue 105. In another preferred embodiment, the interference nubs 106 interface with the external housing 30 such that the internal-housing tongues 105 are biased inward. As shown in FIG. 2, the internal housing 25 nests within the external housing 30 such that their respective opposing sidewalls substantially mate or lie close enough together that the protruding interference nubs 106 interface with the external housing 30, which in turn causes the internal-housing tongues 105 to flex or bias inward toward the lancet-holder 20.

In a further preferred embodiment, the external housing 30 defines a release-window 110 on each opposing sidewall 65a,b. During assembly, as the internal housing 25 enters the distal opening of the external housing 30 and advances toward the proximal end 30a, the interference nubs 106 on the internal-housing tongues 105 will enter the release-windows 110. A tool can be used to block the release-windows 110 (as well as the openings 125 on opposing sidewalls 65a,b, discussed below) during assembly of the apparatus 10 until the internal housing 25 has been advanced to a point where the interference nubs 106 are proximally located relative to the release-windows 110. The function of the release-windows 110 is described below.

In another preferred embodiment, the interference nubs 106 interface with the lancet-holder detents 47 when the internal-housing tongues 105 are biased inward. The interference nubs 106 thereby prevent the lancet-holder 20 from moving proximally until the single handed squeeze lancet apparatus 10 is in use. As pressure is applied to the proximal end 25a of the internal housing 25, the internal housing 25 begins to advance distally within the external housing 30. The interference nubs 106 on the internal housing 25 translate that distal movement to the detents 47 and, in turn, to the lancet-holder 20, which subsequently pre-loads the thumb-spring 40. The thumb-spring 40 continues to pre-load until the interference nubs 106 of the internal housing 25 enter the release-windows 110 of the external housing 30, which unbiases the internal-housing tongues 105 and eliminates the interface between the detents 47 and the interference nubs 106. As the interference nubs 106 travel the length of the release-windows 110, the thumb-spring 40 rapidly moves the lancet-holder 20 proximally and the lancet 15 pierces the skin of the digit applying pressure to the proximal end 25a of the internal housing 25. After the lancet-holder 20 and lancet 15 are released by the interference nubs 106, the interference nubs 106 bottom-out on the distal side of the release-windows 110, halting the distal movement of the internal housing 25. The interference nubs 106 are now trapped in the release-windows 110 preventing significant movement of the internal housing 25 relative to the external housing 30. Similarly, the thumb-spring 40 is now in a static position, which prevents the lancet 15 from extending beyond the proximal end 25a of the internal housing 25.

Alternatively, in another embodiment shown in FIGS. 10-11, the interference nubs 106′ snap-fit into the release windows '110. The thumb-spring 40′ then rapidly moves the lancet-holder 20′ proximally until the lancet-holder 20′ contacts depth-control ribs 56′ molded into the internal housing's front and back walls 55′, 60′ on either side of the internal housing's proximal opening. The length of the depth-control ribs 56′ determine the maximum depth penetration of the lancet into the digit; this depth is in the range of 0.01 to 0.2 inches and is preferably approximately 0.06 inches.

In one preferred embodiment, a proximal end 25a of the internal housing 25 defines an indentation 115 for receiving a user's digit. The indentation 115 preferably takes the form of an arcuate notch, though other reliefs, such as a squared off, or stepped, relief with multiple flat surfaces, are also contemplated.

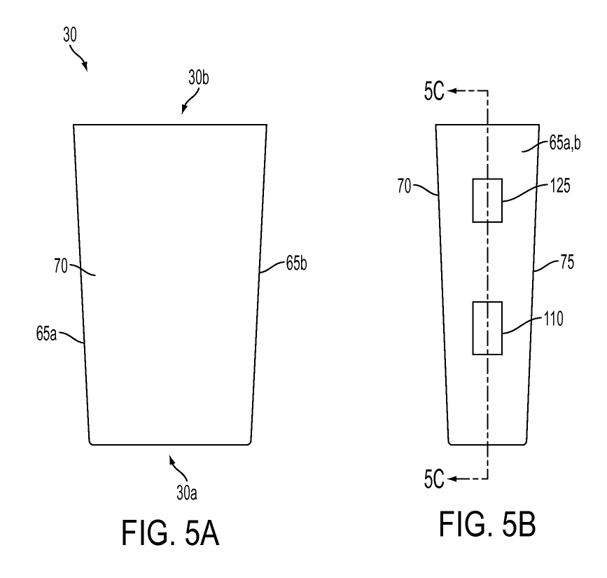

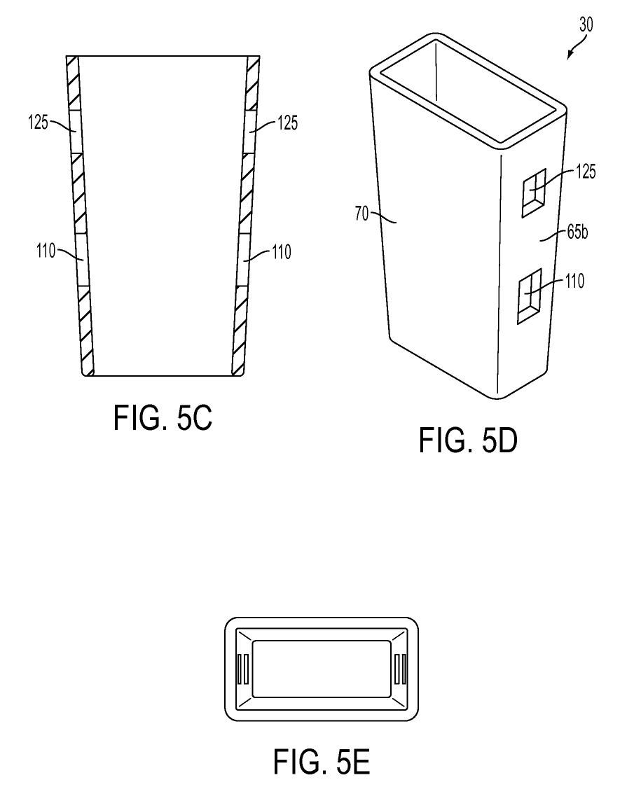

In another preferred embodiment, the internal housing 25 and external housing 30 each taper from the distal end 25b, 30b to the proximal end 25a, 30a, as shown in FIGS. 5A and 6A. The internal housing's opposing sidewalls 50a,b and front 55 and back walls 60 taper vertically inward at an angle less than or equal to 18 degrees, preferably at an angle in the range of 0.5 to 3.5 degrees. Similarly, the external housing's opposing sidewalls 65a,b and front 70 and back walls 75 preferably vertically taper inward at an angle less than or equal to 18 degrees, preferably at an angle in the range of 0.5 to 3.5 degrees. In a further preferred embodiment, the internal housing 25 and external housing 30 substantially mate such that the internal housing 25 is prevented from rotating within the external housing 30 and such that the proximal end 25a of the internal housing 25 is capable of protruding from the proximal end 30a of the external housing 30 but cannot pass all the way through the proximal opening of the external housing. As discussed above, the internal housing 25 protrudes from the external housing 30 a predetermined distance to allow the thumb-spring 40 to become sufficiently pre-loaded as the internal housing 25 is advanced distally.

In one preferred embodiment, the thumb-frame 35 is coupled to two spaced-apart tongues 120, wherein each thumb-frame tongue has a detent 121 at the free end, and wherein the external housing 30 defines openings 125 on opposing sidewalls 65a,b. The openings 125 on the opposing sidewalls 65a,b of the external housing 30 are located near the housing's distal end 30b to receive the detents 121 from the thumb-frame tongues 120 during assembly.

In another embodiment, the thumb-frame 35 is in the form of a base (not shown), such that the single handed squeeze lancet 10 is employed between a digit and a surface. As used herein, the distal end of the thumb-frame 35 provides a planar surface that stabilizes the single handed squeeze lancet 10 when the apparatus is employed between a digit and a surface. This planar surface may take any number of shapes, for example, a square, a rectangle, a circle, or a cross, as long as the surface provides enough stability to prevent the apparatus 10 from tipping over or sliding away from the user's digit.

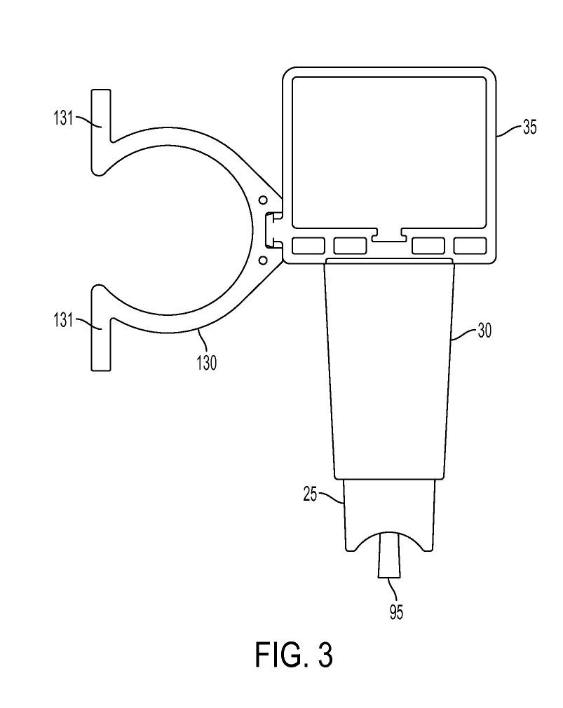

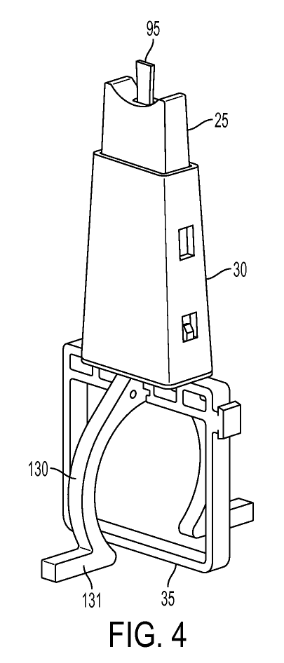

In one embodiment, the single handed squeeze lancet apparatus 10 may further comprise a base-stand 130, shown in FIGS. 3-4. As used herein, the base-stand 130 is coupled to the distal end of the single handed squeeze lancet apparatus 10 to allow operation with a single digit. In a further preferred embodiment, the base-stand 130 is removably coupled to one of the thumb-frame 35 or the external housing 30. Specifically, the base-stand 130 could be coupled to the exterior of either the external housing 30 or thumb-frame 35 or to the interior of the thumb-frame 35 via one or more flanges 135 or other male/female connectors. Alternatively, the base-stand 130 could be molded as one part with either the external housing 30 or thumb-frame 35 with a weak bridge such that the base-stand 130 could be twisted or cut apart for use. This embodiment allows the lancet apparatus 10 to maintain a substantially flat, compact, low profile design for shipping and storage purposes. The base-stand 130 can be of any suitable shape. For example, as shown in FIGS. 3-4, the base-stand 130 can take the form of an Omega frame terminating in perpendicular stabilizers 131. Other possible base-stand shapes include, but are not limited to, a U-Shape or a V-shape terminating in perpendicular stabilizers 131, and, in the case where there is a partial thumb-frame (not shown) with an open distal end, a base-stand 130 with a single perpendicular stabilizer connecting both sides of the frame.

In one preferred embodiment, the base-stand 130 is coupled to the thumb-frame 35 such that the base-stand 130 and thumb-frame 35 together form a base, whereby the single handed squeeze lancet apparatus 10 is employed between a digit and a surface. Here, the base-stand 130 and thumb-frame 35 are substantially perpendicular to each other, as shown in FIG. 4. The base-stand 130 and thumb-frame 35 are dimensioned such that, when they are coupled together, their distal surfaces lie in substantially the same plane. In this embodiment, the thumb-frame 35 preferably defines a square or rectangular outer profile to maximize the surface area of the base, thereby increasing stability. However, a partial thumb-frame may be employed in this embodiment, especially where the distal ends terminate in perpendicular stabilizers 131, as shown for example with regard to the base-stand 130 in FIG. 4. Alternatively, the base-stand 130 may be coupled to the external housing 30.

As shown in FIGS. 2-3, in another preferred embodiment, the thumb-frame 35 defines a detent 140 for receiving a corresponding thin-walled section of the base-stand 130. Alternatively, the base-stand 130 may define a detent to receive a corresponding section of the thumb-frame 35. In addition, if the base stand is connected to the interior of the thumb-frame 35, the base-stand 130 may be rotated 90 degrees about a flange, for example, and into position for lancing a digit. The lancet apparatus 10 contemplates that the thumb-frame 35 and base-stand 130 can be coupled together via any male/female connectors known in the art. And the base-stand 130 may be coupled to the external housing 30 in a similar manner.

In a second aspect, present invention provides a method for lancing a digit using the single handed squeeze lancet apparatus of the first aspect of the invention, where the method comprises: (a) placing a digit adjacent to a proximal end of the internal housing 25 and (b) applying force the proximal end of the internal housing 25.

In one embodiment, the method further comprises: (a) placing a digit from one of the same hand or the other hand adjacent to the thumb-frame 35 and (b) applying force to the thumb-frame 35. As discussed above, applying pressure to the internal housing 25 and thumb-frame 35 activates the lancet apparatus 10. After activation, the user simply discards the disposable lancet apparatus 10 which retains the lancet 15 within internal housing 25 to prevent subsequent accidental piercing. The methods of the invention can be used to draw blood from any subject in need of having blood drawn, including but not limited to diabetic subjects.

In one preferred embodiment, the method further comprises the step of removing a covering from the lancet-holder 20 to expose the lancet 15. As used herein, a “covering” may take the form of heat-sealed packaging or a pull-tab 95 that is either integral or separate from the lancet-holder 20.

In another preferred embodiment, the step of applying force causes: (a) the internal housing 25 to advance distally within the external housing 30, (b) the thumb-spring 40 to preload until interference nubs 106 on the free ends of inwardly biased tongues 105 on opposing walls 50a,b of the internal housing 25 enter release-windows 110 defined by the external housing 30, (c) the lancet 15 to advance proximally until it lances the digit adjacent to the proximal end 25a of the internal housing 25, and (d) the internal housing 25 to continue to advance until the interference nubs 106 travel the length of the release-windows 110.

In one preferred embodiment, the method further comprises the step of retaining the lancet 15 inside the internal housing 25 after the digit is lanced.

In one embodiment, the present invention further provides a method for lancing a digit using the single handed squeeze lancet apparatus 10, where the method comprises: (a) providing the thumb-frame 35 in the form of a base, (b) placing the thumb-frame 35 against a surface, (c) placing a digit adjacent to a proximal end of the internal housing 25, and (d) applying force to the proximal end of the internal housing 25.

In one embodiment, the present invention provides a method for lancing a digit using the single handed squeeze lancet apparatus 10, where the method comprises: (a) providing a base-stand 130, (b) removing the base-stand 130 from a coupling to one of the thumb-frame 35 or the external housing 30, (c) coupling the base-stand 130 to one of the thumb-frame 35 or the external housing 30 to form a base, (d) placing the base against a surface, (e) placing a digit adjacent to the proximal end of the internal housing 25, and (f) applying force to the proximal end of the internal housing 25.

All embodiments of the single handed squeeze lancet of the invention can be used in the methods of the second and third aspects of the invention.

Note that any of the foregoing embodiments of any aspect may be combined together to practice the claimed invention.

1. A single handed squeeze lancet apparatus comprising:
a lancet;
a lancet-holder;
an internal housing for receiving and guiding the lancet-holder;
an external housing for receiving and guiding the internal housing;
a thumb-frame coupled to the external housing; and
a thumb-spring coupled to the thumb-frame and coupled to the lancet-holder.

2. The apparatus of claim 1, wherein the lancet-holder comprises a housing, at least two guide pins, and at least two detents.

3. The apparatus of claim 2, wherein the internal housing defines channels for receiving the lancet-holder guide pins.

4. The apparatus of claim 1, wherein the lancet-holder further comprises a pull-tab.

5. The apparatus of claim 1, wherein at least a portion of the lancet holder's proximal end extends between either a front and a back wall of the internal housing or between guiding ribs.

6. The apparatus of claim 1, wherein the internal housing includes tongues on opposing sidewalls, wherein each internal-housing tongue includes an interference nub on a free end.

7. The apparatus of claim 6, wherein the interference nubs interface with the external housing such that the internal-housing tongues are biased inward.

8. The apparatus of claim 6, wherein the interference nubs interface with the lancet-holder detents when the internal-housing tongues are biased inward.

9. The apparatus of claim 1, wherein the external housing defines a release-window on each opposing sidewall.

10. The apparatus of claim 1, wherein a proximal end of the internal housing defines an indentation for receiving a user's digit.

11. The apparatus of claim 1, wherein the internal housing and external housing each taper from the distal end to the proximal end.

12. The apparatus of claim 1, wherein the internal housing and external housing substantially mate such that the internal housing is prevented from rotating within the external housing and such that the proximal end of the internal housing is capable of protruding from the proximal end of the external housing.

13. The apparatus of claim 1, wherein the thumb-frame is coupled to two spaced-apart tongues, wherein each thumb-frame tongue has a detent at the free end, and wherein the external housing defines openings on opposing sidewalls.

14. The apparatus of claim 1, wherein the thumb-frame is in the form of a base, whereby the single handed squeeze lancet is employed between a digit and a surface.

15. The apparatus of claim 1, further comprising a base-stand.

16. The apparatus of claim 15, wherein the base-stand is removably coupled to one of the thumb-frame or the external housing.

17. The apparatus of claim 15, wherein the base-stand is coupled to the thumb-frame such that the base-stand and thumb-frame together form a base, whereby the single handed squeeze lancet is employed between a digit and a surface.

18. The apparatus of claim 15, wherein the thumb-frame defines a detent for receiving a corresponding thin-walled section of the base-stand.

19. The apparatus of claim 1, wherein the internal housing defines one or more depth-control ribs adjacent to an opening at the proximal end of the internal housing.

20. A method for lancing a digit using the single handed squeeze lancet apparatus of claim 1, the method comprising:
placing a digit adjacent to a proximal end of the internal housing; and
applying force to the proximal end of the internal housing.

21. The method of claim 20 further comprising:
placing a digit from one of the same hand or the other hand adjacent to the thumb-frame; and
applying force to the thumb-frame.

22. The method of claim 20 further comprising:
providing the thumb-frame in the form of a base; and
placing the thumb-frame against a surface.

23. The method of claim 20 further comprising:
providing a base-stand;
removing the base-stand from a coupling to one of the thumb-frame or the external housing;
coupling the base stand to one of the thumb-frame or the external housing to form a base; and
placing the base against a surface.

24. The method of claim 20, further comprising the step of removing a covering from the lancet-holder to expose the lancet.

25. The method of claim 20, wherein the step of applying force causes the internal housing to advance distally within the external housing, the thumb-spring to preload until interference nubs on the free ends of inwardly biased tongues on opposing walls of the internal housing enter release-windows defined by the external housing, the lancet to advance proximally until it lances the digit adjacent to the proximal end of the internal housing, and the internal housing to continue to advance until the interference nubs travel the length of the release-windows.

26. The method of claim 20, wherein the step of applying force causes the internal housing to advance distally within the external housing, the thumb-spring to preload until interference nubs on the free ends of inwardly biased tongues on opposing walls of the internal housing snap-fit into release-windows defined by the external housing, and the lancet to advance proximally until it contacts depth-control ribs to lance the digit adjacent to the proximal end of the internal housing.

27. The method of claim 20, further comprising the step of retaining the lancet inside the internal housing after the digit is lanced.
